(19) United States
(12) Patent Application Publication
(10) Pub. No.: US 2025/0019704 A1
(43) Pub. Date: Jan. 16, 2025

(54) ANTITUMORAL COMPOSITION

(71) Applicants: Mariano Bizzarri, Roma (RM) (IT); AURORA BIOSEARCH SRL, Bollate (MI) (IT)

(72) Inventor: Mariano Bizzarri, Roma (RM) (IT)

(21) Appl. No.: 18/716,054

(22) PCT Filed: Nov. 29, 2022
(86) PCT No.: PCT/IB2022/061511

§ 371 Date: Jun. 3, 2024

(30) Foreign Application Priority Data
Dec. 3, 2021 (IT) 102021000030629

(51) Int. Cl. C12N 15/113; A61K 45/06; A61P 35/00

(52) U.S. Cl. CPC C12N 15/113; A61K 45/06; A61P 35/00; C12N 2310/141

(57) ABSTRACT

The present invention relates to a composition comprising microRNA for use in the treatment of tumours, dietary supplements and/or novel foods and/or drugs that include said composition.

### STATE OF THE ART

In the current era, cancer, together with other chronic degenerative diseases (such as diabetes and neurodegeneration), is one of the major health problems for society. This is a disease linked, among other factors, to longer life expectancy and progressive changes in lifestyles.

Until recently, it was believed that tumours were cells with irreversible damage to the DNA and that the only way to cure it was to eliminate the cancerous cells:
surgically removing them, or
poisoning them with chemotherapy, or
burning them with radiotherapy.

Today, research has found that it is possible to correct and reprogram this DNA damage and restore healthy tumour cells.

This is due to the epigenetics (a branch of science that studies the mechanisms and interactions with which various genes can be regulated, by activating or deactivating them) underlying the phenomenon of tumour reversion.

Tumour reversion provides for an alternative strategy for treating tumours, not by killing diseased cells, but by transforming them into healthy cells. The phenomenon of tumour reversion is first underpinned by organogenesis studies.

Organogenesis is the mechanism for the construction and growth of the various parts of the embryo which meets quantitative and qualitative parameters such that an individual is recognised as belonging to a particular species. The expression organogenesis is used when the embryo has reached the gastrula stage.

In this particular phase, there have been identified particular peptides that can act at epigenetic level for cell differentiation; these mechanisms are precise to an extent that they are capable of identifying and repairing an error as soon as it is intercepted. Should the error be too serious, they are capable of inducing cell apoptosis, therefore eliminating the cell with error. This allows to correct errors induced by carcinogenic products, preventing the formation of tumour.

Advantageously, the present invention relates to a composition of microRNa (miRNA) composition which performs a surprisingly synergistic action in the anti-cancer action in the broad sense. Below are the details thereof.

### Definitions

For the purposes of the present invention, the expression epigenetics is used to indicate the “the branch of biology studies that the causal interaction between genes and their cell product, which bring the phenotype into being” or as “the study of inherited mitotic and meiotic alterations that are not caused by changes in the DNA sequence”. Epigenetic phenomena are, for example, the basis for most cell differentiation processes (and their alteration, therefore also in cancer), and contribute to a certain heritable phenotypic plasticity in relation to environmental changes.

For the purposes of the present invention, the expression “tumour reversion” is used to indicate “reversion of the phenotype of tumour behaviour. This is a process through which the tumour cell is reprogrammed at epigenetic level under the influence of several factors, which may be pharmacological or nutritional, so that the cell no longer behaves like a malignant cell but reacquires the functions and characteristics of a normal cell”.

For the purposes of the present invention, the expression microRNA (miRNA) is used to indicate endogenous single-stranded non-coding RNA molecules observed in the transcriptome of plants, animals and some DNA viruses. These are polymers encoded by eukaryotic nuclear DNA approximately 20-25 nucleotides long and mainly active in the regulation of gene expression at the transcriptional and post-transcriptional level. The miRNAs are incorporated into the RNA-induced silencing complex (RISC) and they induce gene silencing by overlapping with complementary sequences present on target messenger RNA (mRNA) molecules. This link leads to translation suppression or degradation of the target molecule. Silencing may occur according to the following mechanisms:
1. cutting of the mRNA molecule;
2. destabilisation of the mRNA molecule by shortening the poly(A) tail;
3. decrease in the translation efficiency of the messenger;

The human genome encodes hundreds of miRNAs, which are abundant in all mammalian cell types. They perform their silencing activity on a wide range of transcripts derived from the expression of thousands of genes. The aberrant expression of miRNAs is involved in the onset of numerous diseases. They can be used for therapeutic purposes.

For the purposes of the present invention, and according to the Regulation (EU) 2015/2283, which entered into force on 1 Jan. 2018, the expression “novel foods” is used to indicate all foods and substances with no history of “significant” consumption as at 15 May 1997 in the EU, and therefore, they must be approved to evaluate their safety before they can be placed on the market.

For the purposes of the present invention, the miRNA sequences shown here are described in the database https://mirbase.org.

In particular, the miRNA sequences are shown herein with:
the assigned name (e.g. dre-miR-16a); and
accession code (e.g.: MIMAT0001774).

### DETAILED DESCRIPTION OF THE INVENTION

The present invention relates to a composition comprising one or more miRNAs for use in the treatment of cancer, where said miRNAs are selected from the group consisting of:

dre-miR-16a MIMAT0001774:
UAGCAGCACGUAAAUAUUGGUG;
dre-miR-205-5p MIMAT0001280:
UCCUUCAUUCCACCGGAGUCUG;
dre-miR-205-3p MIMAT0031925:
GAUUUCAGUGGUGUGAAGUGUA;
dre-miR-146a MIMAT0001843:
UGAGAACUGAAUUCCAUAGAUGG;
dre-miR-182-5p MIMAT0001271:
UUUGGCAAUGGUAGAACUCACA;
dre-miR-182-3p MIMAT0001272:
UGGUUCUAGACUUGCCAACUA;
dre-miR-29a MIMAT0001802:
UAGCACCAUUUGAAAUCGGUUA;
dre-miR-183-5p MIMAT0001273:
UAUGGCACUGGUAGAAUUCACUG;
dre-miR-183-3p MIMAT0031921:
UGAAUUACCAAAGGGCCAUAA;
dre-miR-155 MIMAT0001851:
UUAAUGCUAAUCGUGAUAGGGG;
dre-miR-363-5p MIMAT0031992:
CGGGUGGAUGACUCUGCAAUUUU;
dre-miR-363-3p MIMAT0001874:
AAUUGCACGGUAUCCAUCUGUA;
dre-miR-192 MIMAT0001275:
AUGACCUAUGAAUUGACAGCC;
dre-miR-101b MIMAT0001815:
UACAGUACUAUGAUAACUGAAG;
dre-miR-15c MIMAT0003764:
AAGCAGCGCGUCAUGGUUUUC;
dre-miR-103 MIMAT0001816:
AGCAGCAUUGUACAGGGCUAUGA;
dre-miR-210-5p MIMAT0003392:
AGCCACUGACUAACGCACAUUG;
dre-miR-210-3p MIMAT0001281:
CUGUGCGUGUGACAGCGGCUAA;
dre-miR-196b MIMAT0001860:
UAGGUAGUUUCAAGUUGUUGGG;
dre-let-7i MIMAT0001767
UGAGGUAGUAGUUUGUGCUGUU
dre-miR-451 MIMAT0001634:
AAACCGUUACCAUUACUGAGUU;
dre-miR-18c MIMAT0001781:
UAAGGUGCAUCUUGUGUAGUUA;
dre-miR-16b MIMAT0001775:
UAGCAGCACGUAAAUAUUGGAG;
dre-miR-10a-5p MIMAT0001267:
UACCCUGUAGAUCCGAAUUUGU;
dre-miR-10a-3p MIMAT0003391:
CAAAUUCGUGUCUUGGGGAAUA;
dre-miR-429a MIMAT0001624:
UAAUACUGUCUGGUAAUGCCGU;
dre-miR-140-5p MIMAT0001836:
CAGUGGUUUUACCCUAUGGUAG;
dre-miR-140-3p MIMAT0003159:
UACCACAGGGUAGAACCACGGAC;
dre-miR-200a-5p MIMAT0031984:
CAUCUUACCGGACAGUGCUGGA;
dre-miR-200a-3p MIMAT0001861:
UAACACUGUCUGGUAACGAUGU;
dre-miR-146b MIMAT0001844:
UGAGAACUGAAUUCCAAGGGUG;
dre-miR-206-5p MIMAT0031988:
ACAUGCUUCCUUAUAUGCCCAUA;
dre-miR-206-3p MIMAT0001866:
UGGAAUGUAAGGAAGUGUGUGG;
dre-miR-20b-5p MIMAT0001778:
CAAAGUGCUCACAGUGCAGGUAG;
dre-miR-20b-3p MIMAT0031939:
ACUGCAAUGUCUGCACUUCAAGU;
dre-miR-454b MIMAT0001878:
UAGUGCAAUAUUGCUUAUAGGG;
dre-miR-15b-5p MIMAT0001773:
UAGCAGCACAUCAUGGUUUGUA;
dre-miR-15b-3p MIMAT0031936:
CGAAUCAUGAUGUGCUGUCACU;
dre-miR-10d-5p MIMAT0001771:
UACCCUGUAGAACCGAAUGUGUG;
dre-miR-10d-3p MIMAT0003394:
CAGAUUCGGUUUUAGGGGAGUA;
dre-miR-10c-5p MIMAT0001770:
UACCCUGUAGAUCCGGAUUUGU;
dre-miR-10c-3p MIMAT0031935:
AAAUUCGUAUCUAGGGGAGUA;
dre-miR-203a-5p MIMAT0031923:
AGUGGUUCUUAACAGUUCAACAGU;
dre-miR-203a-3p MIMAT0001278:
GUGAAAUGUUUAGGACCACUUG;
dre-miR-122 MIMAT0001818:
UGGAGUGUGACAAUGGUGUUUG;
dre-miR-101a MIMAT0001814:
UACAGUACUGUGAUAACUGAAG;
dre-miR-130b MIMAT0001827:
CAGUGCAAUAAUGAAAGGGCAU;
dre-miR-223 MIMAT0001290:
UGUCAGUUUGUCAAAUACCCC;
dre-miR-26b MIMAT0001795:
UUCAAGUAAUCCAGGAUAGGUU;
dre-miR-130a MIMAT0001826:
CAGUGCAAUGUUAAAAGGGCAU;
dre-miR-30e-5p MIMAT0001807:
UGUAAACAUCCUUGACUGGAAG;
dre-miR-30e-3p MIMAT0003402:
CUUUCAGUCGGAUGUUUGCAGC;
dre-miR-203b-5p MIMAT0003407:
AGUGGUUCUCAACAGUUCAACA;
dre-miR-203b-3p MIMAT0001865:
GUGAAAUGUUCAGGACCACUUG;
dre-miR-7b MIMAT0001265:
UGGAAGACUUGUGAUUUUGUU;
dre-miR-221-5p MIMAT0031926:
ACCUGGCAUACAAUGUAGAUUUCUGU;
dre-miR-221-3p MIMAT0001288:
AGCUACAUUGUCUGCUGGGUUUC;
dre-miR-222a-5p MIMAT0031927:
UGCUCAGUAGUCAGUGUAGAUCC;
dre-miR-222a-3p MIMAT0001289:
AGCUACAUCUGGCUACUGGGUCUC;
dre-miR-25-5p MIMAT0031946:
AGGCGGAGACUUGGGCAGCUGCC;
dre-miR-25-3p MIMAT0001793:
CAUUGCACUUGUCUCGGUCUGA;
dre-miR-19b-5p MIMAT0003399:
AGUUUUGCUGGUUUGCAUUCAG;
dre-miR-19b-3p MIMAT0001783:
UGUGCAAAUCCAUGCAAAACUGA;
dre-miR-93 MIMAT0001810:
AAAAGUGCUGUUUGUGCAGGUA;
dre-miR-18a MIMAT0001779:
UAAGGUGCAUCUAGUGCAGAUA;
dre-miR-92a-5p MIMAT0031953:
AGGUUGGGAUUGGUAGCAAUGCU;
dre-miR-92a-3p MIMAT0001808:
UAUUGCACUUGUCCCGGCCUGU;
dre-miR-20a-5p MIMAT0001786:
UAAAGUGCUUAUAGUGCAGGUAG;
dre-miR-20a-3p MIMAT0003400:
ACUGCAGUGUGAGCACUUGAAG;
dre-miR-196c MIMAT0011298:
UAGGUAGUUUGAUGUUGUUGGG;
dre-miR-725-5p MIMAT0032010:
UGCUAGGAAUGGUGGCUGAGAU;
dre-miR-725-3p MIMAT0003753:
UUCAGUCAUUGUUUCUAGUAGU;
dre-miR-733 MIMAT0003763:
UGCGUUGGUUUAGCUCAGUGGUU;

and the mixtures thereof;
and where said cancer is selected from the group consisting of: breast, colon, pancreas and liver cancer.

Preferably, in said composition comprising one or more miRNAs, said miRNAs are selected from the group consisting of:

dre-miR-146a MIMAT0001843:
UGAGAACUGAAUUCCAUAGAUGG;
dre-miR-192 MIMAT0001275:
AUGACCUAUGAAUUGACAGCC;
dre-miR-451 MIMAT0001634:
AAACCGUUACCAUUACUGAGUU;
dre-miR-203b-5p MIMAT0003407:
AGUGGUUCUCAACAGUUCAACA;
dre-miR-203b-3p MIMAT0001865:
GUGAAAUGUUCAGGACCACUUG;
dre-miR-203a-5p MIMAT0031923:
AGUGGUUCUUAACAGUUCAACAGU;
dre-miR-203a-3p MIMAT0001278:
GUGAAAUGUUUAGGACCACUUG;
dre-miR-7b MIMAT0001265:
UGGAAGACUUGUGAUUUUGUU;
dre-miR-19b-5p MIMAT0003399:
AGUUUUGCUGGUUUGCAUUCAG;
dre-miR-19b-3p MIMAT0001783:
UGUGCAAAUCCAUGCAAAACUGA;
dre-miR-725-5p MIMAT0032010:
UGCUAGGAAUGGUGGCUGAGAU;
dre-miR-725-3p MIMAT0003753:
UUCAGUCAUUGUUUCUAGUAGU;

and mixtures thereof.

Preferably, said cancer is breast cancer.

Advantageously, and as shown in the experimental part, the miRNA composition of the present invention may be selected and extracted from Zebrafish; even more preferably, said composition extracted and selected from Zebrafish was selected in the gastrulation stage F6.

Zebrafish extracts (EF) have shown on different types of cultured tumour cells to inhibit tumour motility and invasiveness, triggering apoptotic processes in certain cell fractions and promoting cell reprogramming in the residual samples.

Preferably, said composition of said composition of the present invention may be administered alone or combined with chemotherapy agents. When administered combined with chemotherapy agents, the patients show fewer side effects, leading to an improvement in oncology treatments. The combination with chemotherapy agents actually leads to a decrease in side effects and it increases the tolerability of treatments; furthermore, said composition directly contributes to tumour regression and it improves the overall well-being of patients and their survival.

Advantageously, the anti-cancer activity of the composition of the present invention shows a higher and synergistic action with respect to that o the individual miRNAs.

Advantageously, and as reported in the experimental part, in vitro studies have shown that the miRNA composition of the present invention is capable of promoting a reversion of cancer cells which regain a normal phenotype, losing some of the most important traits of neoplastic cells including the ability to metastasise.

The specific functional configuration of the miRNAs of the present invention allows to trigger in the tested tumour cells an overall differentiation effect with loss of malignancy. This action goes far beyond promoting apoptosis and slowing down proliferation.

Advantageously, and as reported in the experimental part, the composition of the present invention promotes the phenotypic conversion of tumour cells. The processes triggered and controlled by such composition comprise the following mechanisms:
Remodulation of the cytoskeleton configuration resulting in a change in the functional and biophysical relationships between cells and the microenvironment;
Recovery of connections between cells as a prerequisite for the reconstitution of an orderly tissue geometry (re-expression of E-cadherin and cell junctions and in particular E-cadherin/β-catenin transmembrane connections).
Selective modulation of several genes involved in differentiation and in particular inhibition of the expression of TCTP, one of the main genes involved in tumour de-differentiation. It is of great importance that these changes result in the re-expression of p53, considered to be an essential step in regaining control over the cell proliferation mechanisms.
Together with the activation of differentiation processes, treatment with embryonic factors leads to a specific inhibition of the epithelial-mesenchymal transition, with recovery of the epithelial phenotype and inhibition of the mechanisms controlling the triggering of migration and invasiveness (metastatization).

In another embodiment, the present invention relates to dietary supplements and/or novel foods and/or drugs comprising the composition of the present invention.

Below are some descriptive and non-limiting examples aimed at corroborating the inventive and advantageous aspects of the present invention.

The techniques reported in the experimental part are generally used by the person skilled in the art on a routine basis and therefore, where not indicated, the technical information required to reproduce the experiments is believed to be common knowledge for the person skilled in the art the branch technician.

### Examples

1. Extraction of miRNA from Zebrafish

The miRNA content analysis was carried out on six different Zebrafish embryo samples (stages 1-6), corresponding to different stages of embryonic development pre-gastrulation times.

In detail, 516 Zebrafish embryonic cells were stored for each stage:
blastula period (F1);
80% epiboly (F2),
tail (F3),
during the Gastrula period;
10 somite stage (F4),
18 somite stage (F5) and
20 somite stage (F6),

Corresponding to the segmentation period, according to the Zebrafish embryo development stages.

### Principle and Procedure

Mature miRNAs are approximately 22-nucleotide non-coding RNAs present in nature that mediate post-transcriptional gene regulation.

Unlike most cellular RNAs, mature miRNAs have both a 3′ hydroxyl group and a 5′ phosphate group. This allows the adapters to be specifically bound both to the 3′ ends and 5′ ends of the miRNA allowing universal reverse transcription and preparation of the mature miRNA library, reducing the background of other RNA species to the minimum. Furthermore, the QIAseq miRNA Library Kit (QUIAGEN) enables the preparation of the library and the multiplexing of:
a maximum of twelve samples using QIAseq miRNA NGS 12 Index IL,
up to 48 samples combined with QIAseq miRNA NGS 48 Index IL or
up to 96 samples with QIAseq miRNA NGS 96 Index IL.
   Universal Synthesis of cDNA and Preparation of the miRNA Library

In an impartial reaction, the adapters are sequentially bound to the 3′ and 5′ ends of the miRNAs. Subsequently, universal CDNA synthesis (complementary DNA) is carried out with UMI assignment, cDNA clean-up, library amplification, and library clean-up.

The methodology uses modified oligonucleotides and virtually eliminates the presence of adapter dimers in the sequencing library, effectively removing a major contaminant, often observed during sequencing. Furthermore, the kit reduces the presence of hY4 Y-RNA (hY4, small non-coding RNA segments) to the minimum, which is often observed at high levels in serum and plasma samples. The following reactions are part of the workflow:
3′ ligation: A pre-adenylated DNA adapter is ligated to the 3′ ends of all miRNAs. The QIAseq miRNA NGS 3′ Ligase is highly optimised for efficient ligation and prevention of unwanted by-products.
5′ ligation: An RNA adapter is ligated to the 5′ end of the mature miRNAs.
   Preparing the Library for miRNA Sequencing Using the QIAseq miRNA Library Kit.

Specially designed 3′ and 5′ adapters are ligated to mature miRNAs. The ligated miRNAs are then transcribed to cDNA using a reverse transcription primer (RT) with a UMI. No library is prepared by the adapter dimmers.

After cleaning up the cDNA following a standard protocol known to the person skilled in the art, the library is amplified with a universal forward primer and reverse indexing primer.

After a final library clean-up, the miRNA library is therefore ready for quality control and the subsequent Next-Generation Sequencing (NGS).

### CDNA Synthesis

The reverse transcription (RT) primer contains an integrated UMI.

The RT primer binds to a region of the 3′ adapter and facilitates the conversion of 3′/5′ ligated miRNAs to cDNA by assigning A UMI to each miRNA molecule. During reverse transcription, there is also added a universal sequence which is recognised by sample indexing primers during library amplification

### CDNA Clean-Up

After reverse transcription, the cDNA is cleaned up using a simplified magnetic bead-based method, following a standard protocol known to the person skilled in the art.

### Amplifying the Library

The library is amplified using one of the two formats.

In format 1, a universal forward primer wet by a test tube is paired with 1 of the 48 reverse primers wet by test tubes (cat. no. 331592 and 331595) to assign a unique index to each sample. In format 2, a universal forward primer dried by a plate is coupled with 1 of the 96 reverse primers dried in the same plate (cat. no. 331565) to assign a unique custom index to each sample.

In format 2, library amplification reactions occur directly in the index plate, providing a convenient HT indexing solution. The impartial amplification of all miRNAs in a single reaction ensures that there is a sufficient target for next-generation sequencing.

### Library Clean-Up

After amplifying the library, the miRNA library is cleaned up using a simplified magnetic bead-based method, following a standard protocol known to the person skilled in the art.
Input: 100 ng RNA
Sequencing platform: HiSeq 3000 1×75 (SE)

### Results

Contamination Analysis
Distribution of Sequences According to their Lengths. Focus on 17-37-Nucleotide Sequences.
Sequence Complexity Analysis (M=Millions of Sequences)
Sequence Quality Analysis

The identified sequences were divided as miRNA, RNA, and unknown.

The microRNA sequences were therefore aligned to create the count matrix: both a narrow analysis and a wider analysis were carried out. The narrow analysis showed that the sequences having the best alignment with the Zebrafish genome. Sequences corresponding to microRNA were highlighted, as shown below

Distribution of miRNAs in the various
Zebrafish embryo development stages

| Stage | Stage | Stage | Stage | Stage | Stage | |
|---|---|---|---|---|---|---|
| 2 | 3 | 4 | 4b | 5 | 6 | |
| 514 | 460 | 549 | 706 | 193 | 58 | dre-miR-16a |
| 0 | 1408 | 5167 | 3171 | 1492 | 295 | dre-miR-205 |
| 749 | 93 | 57 | 95 | 26 | 0 | dre-mir-146a |
| 0 | 36 | 447 | 312 | 78 | 17 | dre-mir-182 |
| 557 | 3 | 13 | 11 | 9 | 3 | microRNA 29a |
| 11 | 327 | 535 | 337 | 102 | 22 | dre-mir-181c |
| 0 | 20 | 345 | 320 | 71 | 23 | dre-mir-183 |
| 165 | 70 | 154 | 57 | 23 | 9 | dre-mir-155 |
| 196 | 92 | 188 | 177 | 34 | 7 | microRNA 363 |
| 1067 | 97 | 88 | 64 | 14 | 0 | dre-mir-192 |
| 0 | 51 | 43 | 44 | 6 | 0 | dre-mir-101b |
| 21 | 433 | 471 | 237 | 129 | 18 | dre-mir-15c |
| 1969 | 59 | 51 | 55 | 62 | 4 | dre-mir-103 |
| 5 | 19 | 296 | 261 | 116 | 8 | dre-mir-210 |
| 44 | 608 | 2418 | 2150 | 581 | 98 | mir196b |
| 40 | 93 | 145 | 280 | 98 | 37 | dre-let-7i |
| 19715 | 49 | 85 | 220 | 71 | 26 | dre-mir-451 |
| 73 | 168 | 684 | 493 | 107 | 14 | dre-mir-18c |
| 229 | 2550 | 4502 | 4594 | 1309 | 318 | dre-mir-16b |
| 323 | 519 | 2287 | 1508 | 515 | 118 | dre-mir-10a |
| 0 | 749 | 1635 | 1233 | 362 | 86 | dre-mir-429a |
| 0 | 118 | 205 | 163 | 68 | 11 | mir140 |
| 3 | 1151 | 1703 | 1106 | 411 | 65 | dre-mir-200a |
| 985 | 30 | 59 | 28 | 22 | 4 | dre-mir-146b |
| 3 | 984 | 1450 | 1087 | 458 | 107 | dre-mir-206-1 |
| 642 | 256 | 681 | 542 | 122 | 28 | dre-mir-20b |
| 3 | 127 | 604 | 636 | 116 | 42 | dre-mir-454b |
| 23 | 15 | 29 | 29 | 87 | 0 | dre-mir-15b |
| 0 | 0 | 176 | 192 | 48 | 7 | dre-mir-10d |
| 2 | 167 | 807 | 454 | 215 | 55 | dre-mir-10c |
| 3443 | 1808 | 2476 | 3215 | 634 | 120 | dre-mir-203a |
| 16933 | 446 | 929 | 1328 | 329 | 106 | dre-mir-122 |
| 1747 | 7 | 27 | 56 | 14 | 9 | dre-mir-101a |
| 101 | 117 | 297 | 285 | 70 | 6 | dre-mir-130b |
| 4933 | 17 | 28 | 80 | 13 | 7 | mir223 |
| 72 | 177 | 465 | 352 | 106 | 25 | dre-mir-26b |
| 0 | 23 | 41 | 61 | 10 | 0 | dre-mir-130a |
| 3 | 302 | 521 | 512 | 146 | 45 | dre-mir-30e-2 |
| 5228 | 17067 | 22964 | 21454 | 6689 | 1379 | dre-mir-203b |
| 2707 | 451 | 693 | 957 | 244 | 34 | dre-mir-7b |
| 0 | 207 | 1012 | 678 | 265 | 65 | dre-mir-221 |
| 0 | 33 | 214 | 251 | 59 | 29 | dre-mir-222a |
| 0 | 12859 | 18137 | 11886 | 4765 | 1172 | dre-mir-25 |
| 3953 | 28 | 198 | 63 | 7 | 0 | dre-mir-19b |
| 2 | 553 | 606 | 466 | 161 | 27 | dre-mir-93 |
| 132 | 81 | 76 | 53 | 10 | 0 | dre-mir-18a |
| 472 | 521 | 333 | 104 | 95 | 0 | dre-mir-92a-1 |
| 867 | 557 | 616 | 729 | 135 | 36 | dre-mir-20a |
| 31 | 86 | 1853 | 1665 | 294 | 56 | mir196c |
| 393 | 2907 | 3849 | 3395 | 864 | 168 | dre-mir-725 |
| 7 | 30 | 49 | 28 | 18 | 7 | dre-mir-733 |

### Biological Activity

The biological anti-cancer activity of extracts from Zebrafish embryo is mainly expressed by the components (miRNA) in stage 5 and 6, although the most significant effects were observed by testing Stage 6 (Proietti S, Cucina A, Pensotti A, et al. Active Fraction from Embryo Fish Extracts Induces Reversion of the Malignant Invasive Phenotype in Breast Cancer through Down-regulation of TCTP and Modulation of E-cadherin/β-catenin Pathway. Int J Mol Sci. 2019; 20 (9): 2151. Published 2019 Apr. 30. doi: 10.3390/ijms20092151).

miRNA isolates at the 20-somite development stage can reverse several malignant characteristics of the carcinogenic phenotype in a human breast cancer model.

Embryo extracts belonging to stage 6 reduce cell proliferation, improve apoptosis, and drastically inhibit both the invasiveness and migration capacity of tumour cells.

The inhibition of migratory and invasive properties is not limited to breast cancer cells, given that embryonic extracts were also effective in inhibiting the migratory phenotype adopted by normal breast cells in the epithelial-mesenchymal transition stage after stimulation with TGF-β1.

In carcinogenic cells, tumour reversion induced by the embryo involves the E-cadherin/β-catenin pathway, cytoskeleton remodelling, as well as TCTP sub-regulation and concomitant increase in levels of p53.

In particular, it has been investigated that the total extraction pool (Synchro level) and some selected stages (5 and 6) may induce selective changes in the miRNA expression pattern in breast cancer cells (MDA-MB-231), in breast cancer cells committed to malignancy, as well as in a neuroblastoma cell line (SK-N-BE).

It was observed that the Synchro level and selected fractions reduce cell proliferation, improve apoptosis, and drastically inhibit both the invasiveness and migration capacity of tumour cells, although with some significant differences. The inhibition of migratory and invasive properties is not limited to breast cancer cells, given that also that embryonic extracts were effective in inhibiting the migrant phenotype adopted by normal breast cells which are subjected to the epithelial-mesenchymal transition after stimulation with TGF-β1.

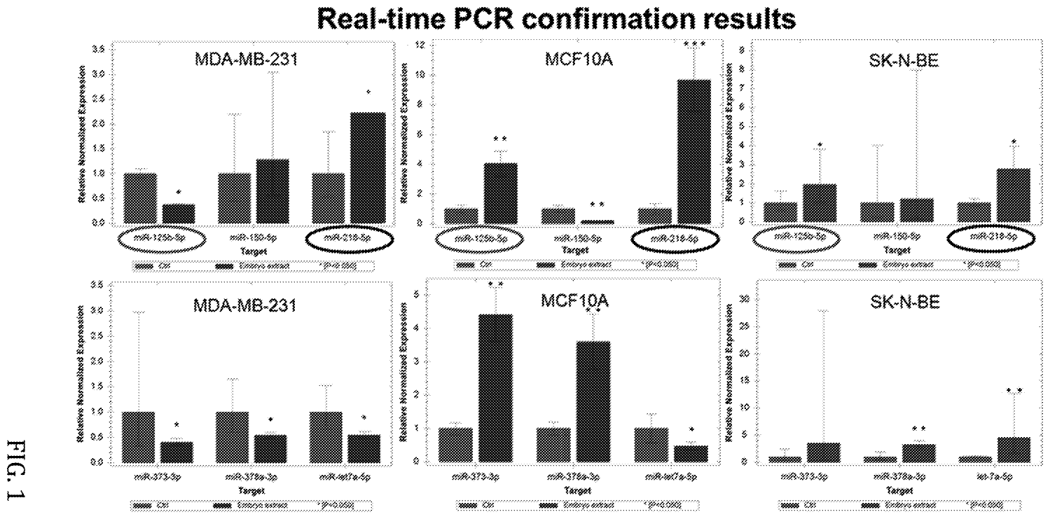

As reported above, in carcinogenic cells, tumour reversion induced by embryonic factors involves the E-cadherin/β-catenin pathway, cytoskeleton remodelling, as well as TCTP down-regulation and concomitant increase in levels of p53. These results show that neoplastic transformation is not an irreversible commitment and it can be “reversed”-even partially in response to the correct stimulation of miRNAs. As a matter of fact, by analysing miRNA expression through miScript™ miRNA PCR Array, differences were found in 84 miRNA released in cell models. Among these, a statistically significant difference was recorded for the following:
a) miR-218-5p (up-regulation);
b) miR-let-7a-5p, miR-378a-3p, miR-150-5p, miR-125b-5p (down-regulation (FIG. 1).

These changes were further confirmed through the RT-PCR study.

Significantly, the aforementioned miRNAs have previously been shown to participate in the control of the motility, invasiveness and progression of the tumour. It has been argued that such changes can successfully inhibit the properties of the tumour, that is the metastatic process.

1. Composition comprising one or more miRNA for use in the treatment of cancer, wherein said one or more miRNA are selected from the group consisting of:
dre-miR-16a MIMAT0001774:
UAGCAGCACGUAAAUAUUGGUG;
dre-miR-205-5p MIMAT0001280:
UCCUUCAUUCCACCGGAGUCUG;
dre-miR-205-3p MIMAT0031925:
GAUUUCAGUGGUGUGAAGUGUA;
dre-miR-182-5p MIMAT0001271:
UUUGGCAAUGGUAGAACUCACA;
dre-miR-182-3p MIMAT0001272:
UGGUUCUAGACUUGCCAACUA;
dre-miR-29a MIMAT0001802:
UAGCACCAUUUGAAAUCGGUUA;
dre-miR-183-5p MIMAT0001273:
UAUGGCACUGGUAGAAUUCACUG;
dre-miR-183-3p MIMAT0031921:
UGAAUUACCAAAGGGCCAUAA;
dre-miR-155 MIMAT0001851:
UUAAUGCUAAUCGUGAUAGGGG;
dre-miR-363-5p MIMAT0031992:
CGGGUGGAUGACUCUGCAAUUUU;
dre-miR-363-3p MIMAT0001874:
AAUUGCACGGUAUCCAUCUGUA;
dre-miR-192 MIMAT0001275:
AUGACCUAUGAAUUGACAGCC;
dre-miR-101b MIMAT0001815:
UACAGUACUAUGAUAACUGAAG;
dre-miR-15c MIMAT0003764:
AAGCAGCGCGUCAUGGUUUUC;
dre-miR-103 MIMAT0001816:
AGCAGCAUUGUACAGGGCUAUGA;
dre-miR-210-5p MIMAT0003392:
AGCCACUGACUAACGCACAUUG;
dre-miR-210-3p MIMAT0001281:
CUGUGCGUGUGACAGCGGCUAA;
dre-miR-196b MIMAT0001860:
UAGGUAGUUUCAAGUUGUUGGG;
dre-let-7i MIMAT0001767
UGAGGUAGUAGUUUGUGCUGUU
dre-miR-451 MIMAT0001634:
AAACCGUUACCAUUACUGAGUU;
dre-miR-18c MIMAT0001781:
UAAGGUGCAUCUUGUGUAGUUA;
dre-miR-16b MIMAT0001775:
UAGCAGCACGUAAAUAUUGGAG;
dre-miR-10a-5p MIMAT0001267:
UACCCUGUAGAUCCGAAUUUGU;
dre-miR-10a-3p MIMAT0003391:
CAAAUUCGUGUCUUGGGGAAUA;
dre-miR-429a MIMAT0001624:
UAAUACUGUCUGGUAAUGCCGU;
dre-miR-140-5p MIMAT0001836:
CAGUGGUUUUACCCUAUGGUAG;
dre-miR-140-3p MIMAT0003159:
UACCACAGGGUAGAACCACGGAC;
dre-miR-200a-5p MIMAT0031984:
CAUCUUACCGGACAGUGCUGGA;
dre-miR-200a-3p MIMAT0001861:
UAACACUGUCUGGUAACGAUGU;
dre-miR-146b MIMAT0001844:
UGAGAACUGAAUUCCAAGGGUG;
dre-miR-206-5p MIMAT0031988:
ACAUGCUUCCUUAUAUGCCCAUA;
dre-miR-206-3p MIMAT0001866:
UGGAAUGUAAGGAAGUGUGUGG;
dre-miR-20b-5p MIMAT0001778:
CAAAGUGCUCACAGUGCAGGUAG;
dre-miR-20b-3p MIMAT0031939:
ACUGCAAUGUCUGCACUUCAAGU;
dre-miR-454b MIMAT0001878:
UAGUGCAAUAUUGCUUAUAGGG;
dre-miR-15b-5p MIMAT0001773:
UAGCAGCACAUCAUGGUUUGUA;
dre-miR-15b-3p MIMAT0031936:
CGAAUCAUGAUGUGCUGUCACU;
dre-miR-10d-5p MIMAT0001771:
UACCCUGUAGAACCGAAUGUGUG;
dre-miR-10d-3p MIMAT0003394:
CAGAUUCGGUUUUAGGGGAGUA;
dre-miR-10c-5p MIMAT0001770:
UACCCUGUAGAUCCGGAUUUGU;
dre-miR-10c-3p MIMAT0031935:
AAAUUCGUAUCUAGGGGAGUA;
dre-miR-203a-5p MIMAT0031923:
AGUGGUUCUUAACAGUUCAACAGU;
dre-miR-203a-3p MIMAT0001278:
GUGAAAUGUUUAGGACCACUUG;
dre-miR-122 MIMAT0001818:
UGGAGUGUGACAAUGGUGUUUG;
dre-miR-101a MIMAT0001814:
UACAGUACUGUGAUAACUGAAG;
dre-miR-130b MIMAT0001827:
CAGUGCAAUAAUGAAAGGGCAU;
dre-miR-223 MIMAT0001290:
UGUCAGUUUGUCAAAUACCCC;
dre-miR-26b MIMAT0001795:
UUCAAGUAAUCCAGGAUAGGUU;
dre-miR-130a MIMAT0001826:
CAGUGCAAUGUUAAAAGGGCAU;
dre-miR-30e-5p MIMAT0001807:
UGUAAACAUCCUUGACUGGAAG;
dre-miR-30e-3p MIMAT0003402:
CUUUCAGUCGGAUGUUUGCAGC;
dre-miR-203b-5p MIMAT0003407:
AGUGGUUCUCAACAGUUCAACA;
dre-miR-203b-3p MIMAT0001865:
GUGAAAUGUUCAGGACCACUUG;
dre-miR-7b MIMAT0001265:
UGGAAGACUUGUGAUUUUGUU;
dre-miR-221-5p MIMAT0031926:
ACCUGGCAUACAAUGUAGAUUUCUGU;
dre-miR-221-3p MIMAT0001288:
AGCUACAUUGUCUGCUGGGUUUC;
dre-miR-222a-5p MIMAT0031927:
UGCUCAGUAGUCAGUGUAGAUCC;
dre-miR-222a-3p MIMAT0001289:
AGCUACAUCUGGCUACUGGGUCUC;
dre-miR-25-5p MIMAT0031946:
AGGCGGAGACUUGGGCAGCUGCC;
dre-miR-25-3p MIMAT0001793:
CAUUGCACUUGUCUCGGUCUGA;
dre-miR-19b-5p MIMAT0003399:
AGUUUUGCUGGUUUGCAUUCAG;
dre-miR-19b-3p MIMAT0001783:
UGUGCAAAUCCAUGCAAAACUGA;
dre-miR-93 MIMAT0001810:
AAAAGUGCUGUUUGUGCAGGUA;
dre-miR-18a MIMAT0001779:
UAAGGUGCAUCUAGUGCAGAUA;
dre-miR-92a-5p MIMAT0031953:
AGGUUGGGAUUGGUAGCAAUGCU;
dre-miR-92a-3p MIMAT0001808:
UAUUGCACUUGUCCCGGCCUGU;
dre-miR-20a-5p MIMAT0001786:
UAAAGUGCUUAUAGUGCAGGUAG;
dre-miR-20a-3p MIMAT0003400:
ACUGCAGUGUGAGCACUUGAAG;
dre-miR-196c MIMAT0011298:
UAGGUAGUUUGAUGUUGUUGGG;
dre-miR-725-5p MIMAT0032010:
UGCUAGGAAUGGUGGCUGAGAU;
dre-miR-725-3p MIMAT0003753:
UUCAGUCAUUGUUUCUAGUAGU;
dre-miR-733 MIMAT0003763:
UGCGUUGGUUUAGCUCAGUGGUU;
and mixtures thereof; and
wherein said cancer is selected from the group consisting of: breast, colon, pancreas, liver cancer.

2. The composition according to claim 1, wherein said one or more miRNAs are selected from the group consisting of:
dre-miR-146a MIMAT0001843:
UGAGAACUGAAUUCCAUAGAUGG;
dre-miR-192 MIMAT0001275:
AUGACCUAUGAAUUGACAGCC;
dre-miR-451 MIMAT0001634:
AAACCGUUACCAUUACUGAGUU;
dre-miR-203b-5p MIMAT0003407:
AGUGGUUCUCAACAGUUCAACA;
dre-miR-203b-3p MIMAT0001865:
GUGAAAUGUUCAGGACCACUUG;
dre-miR-203a-5p MIMAT0031923:
AGUGGUUCUUAACAGUUCAACAGU;
dre-miR-203a-3p MIMAT0001278:
GUGAAAUGUUUAGGACCACUUG;
dre-miR-7b MIMAT0001265:
UGGAAGACUUGUGAUUUUGUU;
dre-miR-19b-5p MIMAT0003399:
AGUUUUGCUGGUUUGCAUUCAG;
dre-miR-19b-3p MIMAT0001783:
UGUGCAAAUCCAUGCAAAACUGA;
dre-miR-725-5p MIMAT0032010:
UGCUAGGAAUGGUGGCUGAGAU;
dre-miR-725-3p MIMAT0003753:
UUCAGUCAUUGUUUCUAGUAGU;
and mixtures thereof.

3. The composition for use according to claim 1, wherein said cancer is breast cancer.

4. The composition for use according to claim 1, wherein said composition is administered alone or combined with chemotherapy agents.

5. A dietary supplement or novel food or drug comprising the composition according to claim 1.

6. The composition for use according to claim 2, wherein said cancer is breast cancer.

7. The composition for use according to claim 6, wherein said composition is administered alone or combined with chemotherapy agents.

8. The composition for use according to claim 3, wherein said composition is administered alone or combined with chemotherapy agents.

9. A dietary supplement or novel food or drug comprising the composition according to claim 2.


SEQUENCE LISTING (73 sequences)

SEQ ID NO: 1 - RNA, 22 nt, Danio rerio
  source 1..22: mol_type = mRNA
tagcagcacgtaaatattggtg

SEQ ID NO: 2 - RNA, 22 nt, Danio rerio
  source 1..22: mol_type = mRNA
tccttcattccaccggagtctg

SEQ ID NO: 3 - RNA, 22 nt, Danio rerio
  source 1..22: mol_type = mRNA
gatttcagtggtgtgaagtgta

SEQ ID NO: 4 - RNA, 23 nt, Danio rerio
  source 1..23: mol_type = mRNA
tgagaactgaattccatagatgg

SEQ ID NO: 5 - RNA, 22 nt, Danio rerio
  source 1..22: mol_type = mRNA
tttggcaatggtagaactcaca

SEQ ID NO: 6 - RNA, 21 nt, Danio rerio
  source 1..21: mol_type = mRNA
tggttctagacttgccaacta

SEQ ID NO: 7 - RNA, 22 nt, Danio rerio
  source 1..22: mol_type = mRNA
tagcaccatttgaaatcggtta

SEQ ID NO: 8 - RNA, 23 nt, Danio rerio
  source 1..23: mol_type = mRNA
tatggcactggtagaattcactg

SEQ ID NO: 9 - RNA, 21 nt, Danio rerio
  source 1..21: mol_type = mRNA
tgaattaccaaagggccataa

SEQ ID NO: 10 - RNA, 22 nt, Danio rerio
  source 1..22: mol_type = mRNA
ttaatgctaatcgtgatagggg

SEQ ID NO: 11 - RNA, 23 nt, Danio rerio
  source 1..23: mol_type = mRNA
cgggtggatgactctgcaatttt

SEQ ID NO: 12 - RNA, 22 nt, Danio rerio
  source 1..22: mol_type = mRNA
aattgcacggtatccatctgta

SEQ ID NO: 13 - RNA, 21 nt, Danio rerio
  source 1..21: mol_type = mRNA
atgacctatgaattgacagcc

SEQ ID NO: 14 - RNA, 22 nt, Danio rerio
  source 1..22: mol_type = mRNA
tacagtactatgataactgaag

SEQ ID NO: 15 - RNA, 21 nt, Danio rerio
  source 1..21: mol_type = mRNA
aagcagcgcgtcatggttttc

SEQ ID NO: 16 - RNA, 23 nt, Danio rerio
  source 1..23: mol_type = mRNA
agcagcattgtacagggctatga

SEQ ID NO: 17 - RNA, 22 nt, Danio rerio
  source 1..22: mol_type = mRNA
agccactgactaacgcacattg

SEQ ID NO: 18 - RNA, 22 nt, Danio rerio
  source 1..22: mol_type = mRNA
ctgtgcgtgtgacagcggctaa

SEQ ID NO: 19 - RNA, 22 nt, Danio rerio
  source 1..22: mol_type = mRNA
taggtagtttcaagttgttggg

SEQ ID NO: 20 - RNA, 22 nt, Danio rerio
  source 1..22: mol_type = mRNA
tgaggtagtagtttgtgctgtt

SEQ ID NO: 21 - RNA, 22 nt, Danio rerio
  source 1..22: mol_type = mRNA
aaaccgttaccattactgagtt

SEQ ID NO: 22 - RNA, 22 nt, Danio rerio
  source 1..22: mol_type = mRNA
taaggtgcatcttgtgtagtta

SEQ ID NO: 23 - RNA, 22 nt, Danio rerio
  source 1..22: mol_type = mRNA
tagcagcacgtaaatattggag

SEQ ID NO: 24 - RNA, 21 nt, Danio rerio
  source 1..21: mol_type = mRNA
tacccgtagatccgaatttgt

SEQ ID NO: 25 - RNA, 22 nt, Danio rerio
  source 1..22: mol_type = mRNA
caaattcgtgtcttggggaata

SEQ ID NO: 26 - RNA, 22 nt, Danio rerio
  source 1..22: mol_type = mRNA
taatactgtctggtaatgccgt

SEQ ID NO: 27 - RNA, 22 nt, Danio rerio
  source 1..22: mol_type = mRNA
cagtggttttaccctatggtag

SEQ ID NO: 28 - RNA, 24 nt, Danio rerio
  source 1..24: mol_type = mRNA
taccaccagggtagaaccacggac

SEQ ID NO: 29 - RNA, 22 nt, Danio rerio
  source 1..22: mol_type = mRNA
catcttaccggacagtgctgga

SEQ ID NO: 30 - RNA, 22 nt, Danio rerio
  source 1..22: mol_type = mRNA
taacactgtctggtaacgatgt

SEQ ID NO: 31 - RNA, 22 nt, Danio rerio
  source 1..22: mol_type = mRNA
tgagaactgaattccaagggtg

SEQ ID NO: 32 - RNA, 23 nt, Danio rerio
  source 1..23: mol_type = mRNA
acatgcttccttatatgcccata

SEQ ID NO: 33 - RNA, 22 nt, Danio rerio
  source 1..22: mol_type = mRNA
tggaatgtaaggaagtgtgtgg

SEQ ID NO: 34 - RNA, 23 nt, Danio rerio
  source 1..23: mol_type = mRNA
caaagtgctcacagtgcaggtag

SEQ ID NO: 35 - RNA, 23 nt, Danio rerio
  source 1..23: mol_type = mRNA
actgcaatgtctgcacttcaagt

SEQ ID NO: 36 - RNA, 22 nt, Danio rerio
  source 1..22: mol_type = mRNA
tagtgcaatattgcttataggg

SEQ ID NO: 37 - RNA, 22 nt, Danio rerio
  source 1..22: mol_type = mRNA
tagcagcacatcatggtttgta

SEQ ID NO: 38 - RNA, 22 nt, Danio rerio
  source 1..22: mol_type = mRNA
cgaatcatgatgtgctgtcact

SEQ ID NO: 39 - RNA, 23 nt, Danio rerio
  source 1..23: mol_type = mRNA
taccctgtagaaccgaatgtgtg

SEQ ID NO: 40 - RNA, 22 nt, Danio rerio
  source 1..22: mol_type = mRNA
cagattcggttttaggggagta

SEQ ID NO: 41 - RNA, 22 nt, Danio rerio
  source 1..22: mol_type = mRNA
taccctgtagatccggatttgt

SEQ ID NO: 42 - RNA, 21 nt, Danio rerio
  source 1..21: mol_type = mRNA
aaattcgtatctaggggagta

SEQ ID NO: 43 - RNA, 24 nt, Danio rerio
  source 1..24: mol_type = mRNA
agtggttcttaacagttcaacagt

SEQ ID NO: 44 - RNA, 22 nt, Danio rerio
  source 1..22: mol_type = mRNA
gtgaaatgtttaggaccacttg

SEQ ID NO: 45 - RNA, 23 nt, Danio rerio
  source 1..23: mol_type = mRNA
tgggagtgtgacaatggtgtttg

SEQ ID NO: 46 - RNA, 22 nt, Danio rerio
  source 1..22: mol_type = mRNA
tacagtactgtgataactgaag

SEQ ID NO: 47 - RNA, 23 nt, Danio rerio
  source 1..23: mol_type = mRNA
cagtgcaataatagaaagggcat

SEQ ID NO: 48 - RNA, 21 nt, Danio rerio
  source 1..21: mol_type = mRNA
tgtcagtttgtcaaatacccc

SEQ ID NO: 49 - RNA, 22 nt, Danio rerio
  source 1..22: mol_type = mRNA
ttcaagtaatccaggataggtt

SEQ ID NO: 50 - RNA, 22 nt, Danio rerio
  source 1..22: mol_type = mRNA
cagtgcaatgttaaaagggcat

SEQ ID NO: 51 - RNA, 22 nt, Danio rerio
  source 1..22: mol_type = mRNA
tgtaaacatccttgactggaag

SEQ ID NO: 52 - RNA, 22 nt, Danio rerio
  source 1..22: mol_type = mRNA
ctttcagtcggatgtttgcagc

SEQ ID NO: 53 - RNA, 22 nt, Danio rerio
  source 1..22: mol_type = mRNA
agtggttctcaacagttcaaca

SEQ ID NO: 54 - RNA, 22 nt, Danio rerio
  source 1..22: mol_type = mRNA
gtgaaatgttcaggaccacttg

SEQ ID NO: 55 - RNA, 21 nt, Danio rerio
  source 1..21: mol_type = mRNA
tggaagacttgtgattttgtt

SEQ ID NO: 56 - RNA, 26 nt, Danio rerio
  source 1..26: mol_type = mRNA
acctggcatacaatgtagatttctgt

SEQ ID NO: 57 - RNA, 23 nt, Danio rerio
  source 1..23: mol_type = mRNA
agctacattgtctgctgggtttc

SEQ ID NO: 58 - RNA, 23 nt, Danio rerio
  source 1..23: mol_type = mRNA
tgctcagtagtcagtgtagatcc

SEQ ID NO: 59 - RNA, 24 nt, Danio rerio
  source 1..24: mol_type = mRNA
agctacatctggctactgggtctc

SEQ ID NO: 60 - RNA, 23 nt, Danio rerio
  source 1..23: mol_type = mRNA
aggcggagacttgggcagctgcc

SEQ ID NO: 61 - RNA, 22 nt, Danio rerio
  source 1..22: mol_type = mRNA
cattgcacttgtctcggtctga

SEQ ID NO: 62 - RNA, 22 nt, Danio rerio
  source 1..22: mol_type = mRNA
agttttgctggtttgcattcag

SEQ ID NO: 63 - RNA, 23 nt, Danio rerio
  source 1..23: mol_type = mRNA
tgtgcaaatccatgcaaaactga

SEQ ID NO: 64 - RNA, 22 nt, Danio rerio
  source 1..22: mol_type = mRNA
aaaagtgctgtttgtgcaggta

SEQ ID NO: 65 - RNA, 22 nt, Danio rerio
  source 1..22: mol_type = mRNA
taaggtgcatctagtgcagata

SEQ ID NO: 66 - RNA, 23 nt, Danio rerio
  source 1..23: mol_type = mRNA
aggttgggattggtagcaatgct

SEQ ID NO: 67 - RNA, 22 nt, Danio rerio
  source 1..22: mol_type = mRNA
tattgcacttgtcccggcctgt

SEQ ID NO: 68 - RNA, 23 nt, Danio rerio
  source 1..23: mol_type = mRNA
taaagtgcttatagtgcaggtag

SEQ ID NO: 69 - RNA, 22 nt, Danio rerio
  source 1..22: mol_type = mRNA
actgcagtgtgagcacttgaag

SEQ ID NO: 70 - RNA, 22 nt, Danio rerio
  source 1..22: mol_type = mRNA
taggtagtttgatgttgttggg

SEQ ID NO: 71 - RNA, 22 nt, Danio rerio
  source 1..22: mol_type = mRNA
tgctaggaatggtggctgagat

SEQ ID NO: 72 - RNA, 22 nt, Danio rerio
  source 1..22: mol_type = mRNA
ttcagtcattgtttctagtagt

SEQ ID NO: 73 - RNA, 22 nt, Danio rerio
  source 1..22: mol_type = mRNA
tgcgttgtttagctcagtggtt